(19) United States
(12) Patent Application Publication
(10) Pub. No.: US 2024/0269426 A1
(43) Pub. Date: Aug. 15, 2024

(54) Intermittent Catheters and Methods

(71) Applicant: C. R. Bard, Inc., Franklin Lakes, NJ (US)

(72) Inventor: Shahab Siddiqui, Lawrenceville, GA (US)

(21) Appl. No.: 18/568,208

(22) PCT Filed: May 16, 2022
(86) PCT No.: PCT/US22/29431

§ 371 Date: Dec. 7, 2023

Related U.S. Application Data
(60) Provisional application No. 63/209,832, filed on Jun. 11, 2021.

(51) Int. Cl. A61M 25/00; A61L 29/08

(52) U.S. Cl. CPC A61M 25/0009; A61L 29/08; A61L 2400/10; A61M 2025/0046; A61M 2210/1092; A61M 2210/1096

(57) ABSTRACT

Disclosed herein are intermittent catheters and methods thereof. In an example of an intermittent catheter, the intermittent catheter can include a catheter tube, a lubricant disposed over a surface of at least a distal portion of the catheter tube, and a funnel coupled to a proximal portion of the catheter tube. The catheter tube can be of a polymeric material. The lubricant can have a solid state below 68° F. and a liquid state above 68° F. such that the lubricant transitions into the liquid state upon insertion into a urethra of a patient or user. The funnel can include a funnel opening in a proximal end of the funnel for voiding urine. In an example of a method of an intermittent catheter, the method can include a method of making or using the intermittent catheter.

### PRIORITY

This application claims the benefit of priority to U.S. Provisional Application No. 63/209,832, filed Jun. 11, 2021, which is incorporated by reference in its entirety into this application.

### BACKGROUND

For urethral insertion of an intermittent catheter, the intermittent catheter generally needs to be lubricated. In one example, a dry intermittent catheter is manually lubricated by application of a lubricant before the urethral insertion. In another example, a water-activated hydrophilic coating-coated intermittent catheter is used for urethral insertion. Water immediately swells the hydrophilic coating upon contact with water and makes for a highly lubricious surface of the intermittent catheter for the urethral insertion. Wetting the water-activated hydrophilic coating of the intermittent catheter is achieved by bursting open a water sachet included in a package holding the intermittent catheter followed by moving the catheter back and forth in the water within the package for prior to the urethral insertion. In yet another example, the intermittent catheter is already wetted in its package for the urethral insertion. In each case of the above cases, the intermittent catheter can be messy to handle during the urethral insertion as well as thereafter such as when disposing of the intermittent catheter. What is needed is an intermittent catheter that is clean and mess-free both before and after urethral insertion.

Disclosed herein are intermittent catheters and methods that address the foregoing.

### SUMMARY

Disclosed herein is an intermittent catheter including, in some embodiments, a catheter tube, a lubricant disposed over a surface of at least a distal portion of the catheter tube, and a funnel coupled to a proximal portion of the catheter tube. The catheter tube is of a polymeric material. The lubricant is configured to transitions from a solid state into a liquid state upon insertion into a urethra of a patient or user. The funnel includes a funnel opening in a proximal end of the funnel for voiding urine.

In some embodiments, the lubricant is disposed over the surface of the catheter tube in a particulate form of the lubricant.

In some embodiments, the lubricant is substantially free of water.

In some embodiments, the lubricant has a melting point between 70° F. and 95° F.

In some embodiments, the lubricant is a natural fat, a synthetic fat, or a combination thereof.

In some embodiments, the lubricant is coconut oil, cocoa butter, palm oil, shea butter, or a mixture thereof.

In some embodiments, the polymeric material is thermoplastic polyurethane (“TPU”), silicone, polyvinyl chloride, or a rubber.

In some embodiments, the surface of the catheter tube is modified by a plasma treatment.

In some embodiments, a surface energy of the polymeric material at the surface of the catheter tube is at least 5 dynes/cm greater than that of the lubricant.

In some embodiments, a surface energy of the polymeric material at the surface of the catheter tube is at least 10 dynes/cm greater than that of the lubricant.

Also disclosed herein is another intermittent catheter including, in some embodiments, a hydrophilic coating disposed over a surface of at least a distal portion of the catheter tube, a lubricant disposed over the hydrophilic coating, and a funnel coupled to a proximal portion of the catheter tube. The catheter tube is of a polymeric material. The lubricant is configured to transition from a solid state into a liquid state upon insertion into a urethra of a patient or user. The funnel includes a funnel opening in a proximal end of the funnel for voiding urine.

In some embodiments, the lubricant is disposed over the hydrophilic coating in a particulate form of the lubricant.

In some embodiments, the lubricant is substantially free of water.

In some embodiments, the lubricant has a melting point between 70° F. and 95° F.

In some embodiments, the lubricant is a natural fat, a synthetic fat, or a combination thereof.

In some embodiments, the lubricant is coconut oil, cocoa butter, palm oil, shea butter, or a mixture thereof.

In some embodiments, a surface energy of a surface of the hydrophilic coating is at least 5 dynes/cm greater than that of the lubricant.

In some embodiments, a surface energy of a surface of the hydrophilic coating is at least 10 dynes/cm greater than that of the lubricant.

Also disclosed herein is a method of making an intermittent catheter. The method includes, in some embodiments, a heating step, a lubricant-disposing step, and a cooling step. The heating step includes heating a reservoir of a lubricant such that the lubricant transitions from a solid state to a liquid state. The lubricant-disposing step includes dipping at least a distal portion of a catheter tube of an intermittent catheter into the reservoir when the lubricant is in the liquid state thereof. The lubricant-disposing step disposes the lubricant over a surface of at least the distal portion of the catheter tube. The cooling step includes allowing the lubricant disposed over the surface of the catheter tube to cool such that the lubricant transitions from the liquid state to the solid state of the lubricant.

In some embodiments, the lubricant has a melting point between 70° F. and 95° F.

In some embodiments, the lubricant is a natural fat, a synthetic fat, or a combination thereof.

In some embodiments, the lubricant is coconut oil, cocoa butter, palm oil, shea butter, or a mixture thereof.

In some embodiments, the method further includes a surface-modifying step. The surface-modifying step includes treating at least the distal portion of the catheter tube with a plasma treatment to modify the surface of the catheter tube before the lubricant-disposing step.

In some embodiments, the catheter tube is of a polymeric material selected from TPU, silicone, polyvinyl chloride, and a rubber.

In some embodiments, the lubricant-disposing step includes allowing the catheter tube to dwell in the lubricant for 5 to 7 seconds.

In some embodiments, the method further includes a withdrawing step. The withdrawing step includes gradually withdrawing the catheter tube from the reservoir after the lubricant-disposing step.

In some embodiments, the method further includes a repeating step. The repeating step includes repeating the lubricant-disposing step until achieving a desired thickness of the lubricant disposed over the surface of the catheter tube.

In some embodiments, the method further includes a hanging step. The hanging step includes hanging the intermittent catheter on a rack for the cooling step.

Also disclosed herein is another method of making an intermittent catheter. The method includes, in some embodiments, a hydrophilic coating-disposing step, a heating step, a lubricant-disposing step, and a cooling step. The hydrophilic coating-disposing step includes disposing a hydrophilic coating over a surface of at least a distal portion of a catheter tube of an intermittent catheter. The heating step includes heating a reservoir of a lubricant such that the lubricant transitions from a solid state to a liquid state. The lubricant-disposing step includes dipping at least the distal portion of the catheter tube into the reservoir when the lubricant is in the liquid state thereof. The lubricant-disposing step disposes the lubricant over the hydrophilic coating. The cooling step includes allowing the lubricant disposed over the hydrophilic coating to cool such that the lubricant transitions from the liquid state to the solid state of the lubricant.

In some embodiments, the lubricant has a melting point between 70° F. and 95° F.

In some embodiments, the lubricant is a natural fat, a synthetic fat, or a combination thereof.

In some embodiments, the lubricant is coconut oil, cocoa butter, palm oil, shea butter, or a mixture thereof.

In some embodiments, the lubricant-disposing step includes allowing the catheter tube to dwell in the lubricant for 5 to 7 seconds.

In some embodiments, the method further includes a withdrawing step. The withdrawing step includes gradually withdrawing the catheter tube from the reservoir after the lubricant-disposing step.

In some embodiments, the method further includes a repeating step. The repeating step includes repeating the lubricant-disposing step until achieving a desired thickness of the lubricant disposed over the hydrophilic coating.

In some embodiments, the method further includes a hanging step. The hanging step includes hanging the intermittent catheter on a rack for the cooling step.

Also disclosed herein is a method of using an intermittent catheter. The method includes, in some embodiments, an obtaining step, a removing step, an inserting step, and a voiding step. The obtaining step includes obtaining a packaged intermittent catheter including an intermittent catheter disposed in an outer packaging. A catheter tube of the intermittent catheter includes a lubricant in a solid state thereof disposed over a surface of at least a distal portion of the catheter tube. The removing step includes removing the intermittent catheter from the outer packaging by a funnel of the intermittent catheter. The inserting step includes inserting the catheter tube into a urethra. The inserting simultaneously transitions the lubricant disposed over the surface of the catheter tube into a liquid state thereof for lubrication of the catheter tube. The voiding step includes voiding urine from a bladder.

In some embodiments, the method further includes a withdrawing step. The withdrawing step includes withdrawing the catheter tube from the urethra after the voiding step. The withdrawing step simultaneously transitions any remaining lubricant disposed over the surface of the catheter tube into the solid state thereof for clean, mess-free disposal of the intermittent catheter. The transition of the lubricant into the solid state is in accordance with exposing the lubricant to an ambient temperature below body temperature.

In some embodiments, the method further includes a placing step and a resealing step. The placing step includes placing the intermittent catheter in the outer packaging. The resealing step includes resealing the intermittent catheter in the outer packaging. The resealing step prevents residual urine leakage from the intermittent catheter.

In some embodiments, the method further includes a disposing step. The disposing step includes disposing of the intermittent catheter.

These and other features of the concepts provided herein will become more apparent to those of skill in the art in view of the accompanying drawings and following description, which describe particular embodiments of such concepts in greater detail.

### DRAWINGS

FIG. 1 illustrates a male intermittent catheter in accordance with some embodiments.

FIG. 2 illustrates a female intermittent catheter in accordance with some embodiments.

FIG. 3 illustrates a method of using the male intermittent catheter in accordance with some embodiments.

FIG. 4 illustrates a method of using the female intermittent catheter in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient or user. Likewise, a “proximal length” of, for example, the catheter includes a length of the catheter intended to be near the clinician when the catheter is used on the patient or user. A “proximal end” of, for example, the catheter includes an end of the catheter intended to be near the clinician when the catheter is used on the patient or user. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient or user when the catheter is used on the patient or user. Likewise, a “distal length” of, for example, the catheter includes a length of the catheter intended to be near or in the patient or user when the catheter is used on the patient or user. A “distal end” of, for example, the catheter includes an end of the catheter intended to be near or in the patient or user when the catheter is used on the patient or user. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

### Intermittent Catheters

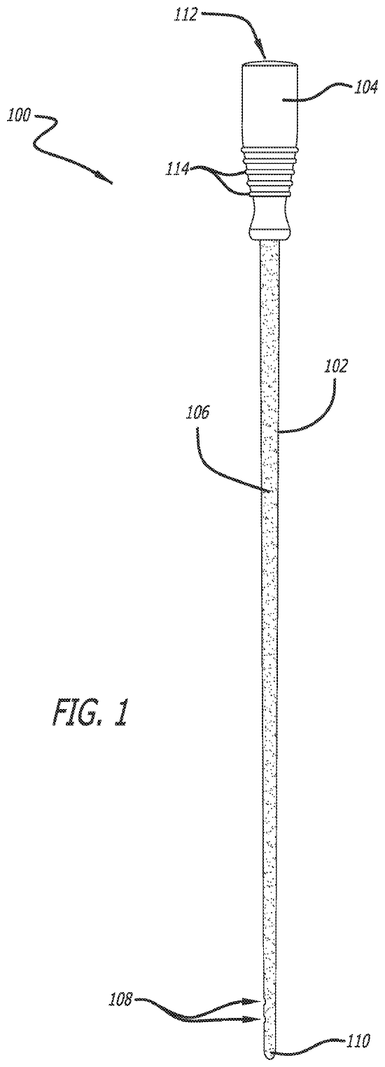

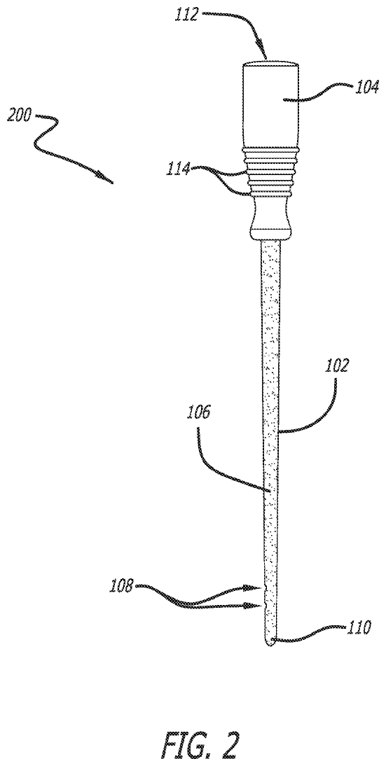

FIGS. 1 and 2 respectively illustrate a male intermittent catheter 100 and a female intermittent catheter 200 in accordance with some embodiments.

As shown, the intermittent catheter 100 or 200 includes a catheter tube 102 or 202 and a funnel 104 coupled to a proximal portion of the catheter tube 102 or 202. The catheter tube 102 or 202 includes a lubricant 106 disposed over a surface of at least a distal portion of the catheter tube 102 or 202 up to an entirety of the catheter tube 102 or 202; however, in some embodiments, the catheter tube 102 or 202 includes a hydrophilic coating disposed over the surface of at least the distal portion of the catheter tube 102 or 202 up to the entirety of the catheter tube 102 or 202 with the lubricant 106 disposed over the hydrophilic coating. Description for the catheter tube 102 or 202 and the funnel 104 are set forth immediately below; description for the lubricant 106 is set forth in the following section.

The catheter tube 102 and the catheter tube 202 differ in that the catheter tube 102 is longer than the catheter tube 202; however, each catheter tube of the catheter tubes 102 and 202 include a plurality of eyelets 108 about a catheter tip 110. The eyelets 108 are in fluid communication with the funnel opening 112 set forth below. In addition, each catheter tube of the catheter tubes 102 and 202 including the surface thereof is of a polymeric material. The polymeric material is TPU, silicone, polyvinyl chloride, or a rubber. The rubber can be a natural rubber, a synthetic rubber, or a combination thereof such as red rubber, which includes a blend of natural rubber and styrene butadiene rubber. Optionally, the surface of the catheter tube 102 or 202 is modified by a plasma treatment to increase a surface energy SE of the polymeric material at the surface of the catheter tube 102 or 202. With or without plasma treatment, the surface energy SE of the polymeric material at the surface of the catheter tube 102 or 202 is at least 5 dynes/cm, 10 dynes/cm, 15 dynes/cm, or 20 dynes/cm greater than that of the lubricant.

The funnel 104 includes a funnel opening 112 that opens in a proximal end of the funnel 104 opposite the catheter tube 102 or 202 for voiding urine. The funnel opening 112 is in fluid communication with the eyelets 108 set forth above. In addition, the funnel 104 includes a plurality of ridges 114 integrated into an outer surface of the funnel 104. The ridges 114 are configured for gripping the funnel 104 as a handle while removing the intermittent catheter 100 or 200 from the outer packaging 116 or 216 of the packaged intermittent catheter 118 or 218 set forth below. Additionally or alternatively, the ridges 114 are configured for gripping the funnel 104 as a handle while inserting the intermittent catheter 100 or 200 into a urethra.

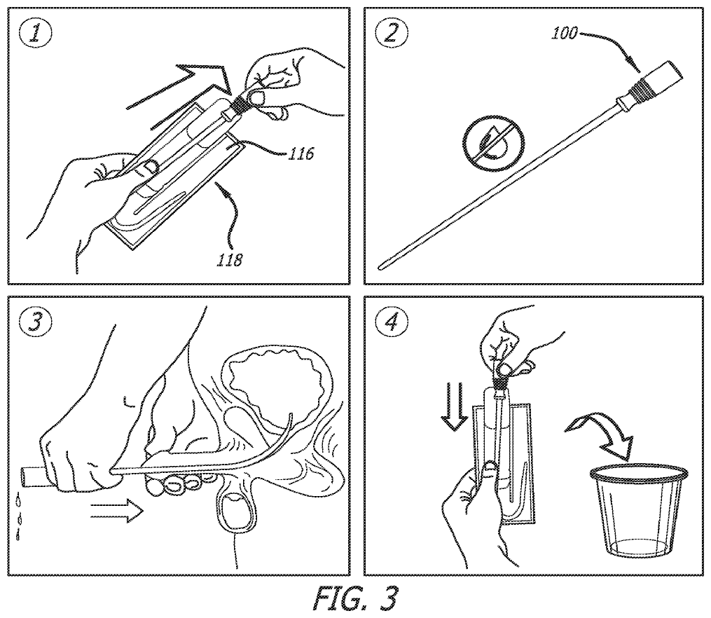

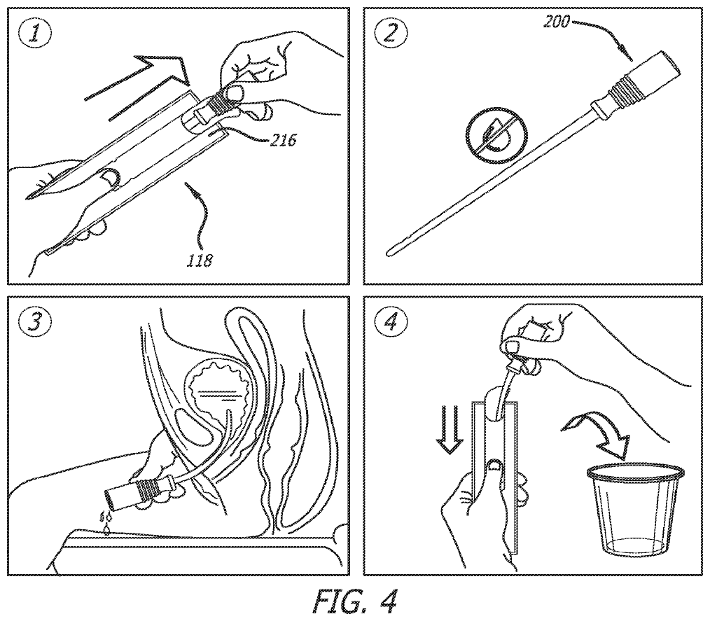

As shown in FIG. 3 or 4 the intermittent catheter 100 or 200 can be disposed in an outer packaging 116 or 216 to form a packaged intermittent catheter 118 or 218. Notably, the intermittent catheter 100 or 200 disposed in the outer packaging 116 or 216 includes the lubricant 106 in a solid state substantially free of water.

### Lubricant

Again, the lubricant 106 is disposed over the surface of at least the distal portion of the catheter tube 102 or 202 up to an entirety of the catheter tube 102 or 202; however, in some embodiments, the lubricant 106 is disposed over the hydrophilic coating, if present, over the surface of the catheter tube 102 or 202.

The lubricant 106 has a solid state below normal temperature and pressure (“NTP”) and a liquid state above NTP, wherein NTP is defined as a temperature of 68° F. and an atmospheric pressure of 760 mmHg per the National Institute of Standards and Technology (“NIST”). Such a lubricant is advantageous in that the lubricant 106 has a solid state below 68° F. such as while the intermittent catheter 100 or 200 is packaged as the packaged intermittent catheter 118 or 218 and stored or immediately before insertion into a urethra. The solid state of the lubricant 106 is also advantageously clean and free from mess in that it can adopt a relatively dry, particulate form over the hydrophilic coating or the surface of the catheter tube 102 or 202, wherein the lubricant 106 is substantially free of water including <1% water, such as <0.5% water, for example, <0.3%, 0.2%, or 0.1% water. The lubricant 106 is further advantageous in that the lubricant 106 has a liquid state above 68° F. such that the lubricant 106 transitions into the liquid state immediately upon insertion into a urethra of a patient or user.

The foregoing lubricant can be a fat such as a natural fat, a semisynthetic fat, a synthetic fat, or a combination thereof having a melting point between 70° F. and 95° F. For example, the lubricant 106 can include, but is not limited to, a natural fat including coconut oil (mp 76° F.), cocoa butter (mp 93° F.), palm oil (mp 95° F.), shea butter (mp 90° F.), or a mixture thereof. A semisynthetic fat including a hydrogenated natural fat such as hydrogenated coconut oil can be used to increase the melting point of the lubricant 106 as desired for warmer environments. It should be understood that while melting points are used herein, melting is a physical change that occurs over a small temperature range of, for example, 2 to 3 degrees Fahrenheit for even the purest materials. Not only is the lubricant 106 highly lubricious in the liquid state thereof, but certain fats such coconut oil are additionally advantageous as the lubricant 106 in that such fats can be beneficial for skin health and the health of organs such as those of the urinary system. Indeed, coconut oil even has antioxidant properties as well as antimicrobial properties, which can help mitigate urinary tract infections (“UTIs”).

The wettability or spreadability of the lubricant 106 (e.g., coconut oil) appears to be governed by the surface energies SEs of the lubricant 106 and the surface of the catheter tube 102 or 202. Such wettability can be understood by consideration of the spreading parameter S of the lubricant 106 in its liquid state, wherein S represents the difference between the work of adhesion Wₐ and the work of cohesion We as in the following equation:

$$S = {W}_{a} - {W}_{c} = {γ}_{S} - ( {γ}_{S L} + {γ}_{L} ) \qquad EQN 1$$

In Equation 1, γₛ is the surface energy of a solid such as the surface of the catheter tube 102 or 202, γ_{L} is the surface tension of a liquid such as the lubricant 106 in the liquid state, and γ_{SL} is the interfacial tension between the solid and liquid. In order for the lubricant 106 in its liquid state to spread over surface of the catheter tube 102 or 202, it is desirable to have S≥0. For exceptional spreading of the lubricant 106 in its liquid state over the surface of the catheter tube 102 or 202, it is desirable to have S≥10. For example, coconut oil has a surface tension of 27.413 dynes/cm in its liquid state. For the coconut oil to adequately wet the surface of the catheter tube 102 or 202, the surface energy SE of the surface of the catheter tube 102 or 202 should be larger than that of the coconut oil by at least 10 dynes/cm or more. If such a condition is not satisfied, however, the surface of the catheter tube 102 or 202 can be treated with a plasma treatment as set forth above, which increases the SE for adequate wetting of the surface of the catheter tube 102 or 202.

### Methods

Methods of the intermittent catheter 100 or 200 include methods of making the intermittent catheter 100 or 200. For example, a method of making the intermittent catheter 100 or 200 can include one or more steps selected from a surface-modifying step, a hydrophilic coating-disposing step, a heating step, a lubricant-disposing step, a withdrawing step, a repeating step, a hanging step, a cooling step, and a packaging step.

When performed, the surface-modifying step includes treating the surface of at least the distal portion of the catheter tube 102 or 202 up to the entirety of the catheter tube 102 or 202 with a plasma treatment to modify the corresponding surface of the of the catheter tube 102 or 202 before performing the hydrophilic coating-disposing step, the lubricant-disposing step, or both. As set forth above, the catheter tube 102 or 202 including the surface of the catheter tube 102 or 202 is of a polymeric material selected from TPU, silicone, polyvinyl chloride, and a rubber.

When performed, the hydrophilic coating-disposing step includes disposing the hydrophilic coating over the surface of at least the distal portion of the catheter tube 102 or 202 up to the entirety of the catheter tube 102 or 202.

The heating step includes heating a reservoir of the lubricant 106 such that the lubricant 106 transitions from a solid state to a liquid state. As set forth above, the solid state of the lubricant 106 is below 68° F. and the liquid state of the lubricant 106 is above 68° F. For example, the lubricant 106 can have a melting point between 70° F. and 95° F., inclusively, such as coconut oil, cocoa butter, palm oil, or shea butter, which have melting points of 76° F., 93° F., 95° F., 90° F., respectively. To ensure the lubricant 106 maintains the liquid state during the heating step and certain subsequent steps such as the lubricant-disposing step, the reservoir of the lubricant 106 can be heated above the melting point of the lubricant 106 such as 5° F., 10° F., 15° F., 20° F., or 25° F. above the melting point of the lubricant 106. For example, if the lubricant 106 is coconut oil, the reservoir of the lubricant 106 can be heated to at least about 100° F.

The lubricant-disposing step includes dipping at least the distal portion of the catheter tube 102 or 202 up to the entirety of the catheter tube 102 or 202 into the reservoir when the lubricant 106 is in the liquid state thereof. The lubricant-disposing step also includes allowing the catheter tube 102 or 202 to dwell in the lubricant 106 for 5 to 7 seconds, thereby providing the lubricant 106 sufficient time to interact with the hydrophilic coating or one or more surface elements (e.g., pores, charged species, etc.) of the surface of the catheter tube 102 or 202. The lubricant-disposing step disposes the lubricant 106 over the hydrophilic coating if the hydrophilic coating is disposed over the surface of the catheter tube 102 or 202. Alternatively, the lubricant-disposing step disposes the lubricant 106 over the surface of the catheter tube 102 or 202 if the hydrophilic coating is not disposed over the surface of the catheter tube 102 or 202.

The withdrawing step includes gradually withdrawing the catheter tube 102 or 202 from the reservoir after the lubricant-disposing step. Notably, the withdrawing step can be viewed as part of the lubricant-disposing step in some embodiments. Regardless, the withdrawing step provides the lubricant 106 nascently disposed over the hydrophilic coating or the surface of the catheter tube 102 or 202 sufficient time to thermally equilibrate with the lubricant 106 in the reservoir at the interface therebetween, which can affect the thickness of the lubricant 106 over the hydrophilic coating or the surface of the catheter tube 102 or 202.

The repeating step includes repeating the lubricant-disposing step and the withdrawing step if not part of the lubricant-disposing step until achieving the thickness of the lubricant 106 over the hydrophilic coating or the surface of the catheter tube 102 or 202 desired. Depending upon the surface energies SEs, the repeating step need not be performed in some embodiments. That said, in other embodiments, the repeating step can be performed any number of times including one time, two times, three times, or more than three times. For example, when the catheter tube 102 or 202 including the surface of the catheter tube 102 or 202 is silicone or PVC, the repeating step can be performed one or times on account of the lower surface energies of silicon and PVC; however, the repeating step need not be performed when the catheter tube 102 or 202 including the surface of the catheter tube 102 or 202 is TPU and the lubricant 106 is coconut oil.

The hanging step includes hanging the intermittent catheter 100 or 200 on a rack for the cooling step.

The cooling step includes allowing the lubricant 106 disposed over the hydrophilic coating or the surface of the catheter tube 102 or 202 to cool such that the lubricant 106 transitions from the liquid state to the solid state of the lubricant 106.

The packaging step includes packaging the intermittent catheter 100 or 200 in the outer packaging 116 or 216.

Methods of the intermittent catheter 100 or 200 include methods of using the intermittent catheter 100 or 200. For example, the method can include one or more steps selected from an obtaining step, a removing step, an inserting step, a voiding step, a withdrawing step, and a placing step.

The obtaining step includes obtaining the packaged intermittent catheter 118 or 218 including the intermittent catheter 100 or 200 disposed in the outer packaging 116 or 216. As set forth above, the intermittent catheter 100 or 200 includes the lubricant 106 disposed over the hydrophilic coating if the hydrophilic coating is disposed over the surface of the catheter tube 102 or 202. Alternatively, the intermittent catheter 100 or 200 includes the lubricant 106 disposed over the surface of the catheter tube 102 or 202 if the hydrophilic coating is not disposed over the surface of the catheter tube 102 or 202.

The removing step includes removing the intermittent catheter 100 or 200 from the outer packaging 116 or 216 by the funnel 104 of the intermittent catheter 100 or 200.

The inserting step includes inserting the catheter tube 102 or 202 into a urethra. The inserting simultaneously transitions the lubricant 106 disposed over the hydrophilic coating or the surface of the catheter tube 102 or 202 into the liquid state thereof for lubrication of the catheter tube 102 or 202. The transition of the lubricant 106 into the liquid state is in accordance with exposing the lubricant 106 to a body temperature above 68° F. such as a body temperature of about 98.6° F.

The voiding step includes voiding urine from a bladder.

The method further includes a withdrawing step. The withdrawing step includes withdrawing the catheter tube 102 or 202 from the urethra after the voiding step. The withdrawing step simultaneously transitions any remaining lubricant 106 disposed over the hydrophilic coating or the surface of the catheter tube 102 or 202 into the solid state thereof for clean, mess-free disposal of the intermittent catheter 100 or 200. The transition of the lubricant 106 into the solid state is in accordance with exposing the lubricant 106 to an ambient temperature below 68° F.

The placing step includes placing the intermittent catheter 100 or 200 in the outer packaging 116 or 216.

The resealing step includes resealing the intermittent catheter 100 or 200 in the outer packaging 116 or 216. The resealing step prevents residual urine leakage from the intermittent catheter 100 or 200.

The disposing step includes disposing of the intermittent catheter 100 or 200.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. An intermittent catheter, comprising:
a catheter tube of a polymeric material;
a lubricant disposed over a surface of at least a distal portion of the catheter tube, the lubricant configured to transition from a solid state to a liquid state upon insertion into a urethra of a patient or user; and
a funnel coupled to a proximal portion of the catheter tube, the funnel including a funnel opening in a proximal end of the funnel for voiding urine.

2. The intermittent catheter of claim 1, wherein the lubricant is disposed over the surface of the catheter tube in a particulate form of the lubricant.

3. The intermittent catheter of claim 1, wherein the lubricant is substantially free of water.

4. The intermittent catheter of claim 1, wherein the lubricant has a melting point between 70° F. and 95° F.

5. The intermittent catheter of claim 1, wherein the lubricant is a natural fat, a synthetic fat, or a combination thereof.

6. The intermittent catheter of claim 1, wherein the lubricant is coconut oil, cocoa butter, palm oil, shea butter, or a mixture thereof.

7. The intermittent catheter of claim 1, wherein the polymeric material is thermoplastic polyurethane (“TPU”), silicone, polyvinyl chloride, or a rubber.

8. The intermittent catheter of claim 1, wherein the surface of the catheter tube is modified by a plasma treatment.

9. The intermittent catheter of claim 1, wherein a surface energy of the polymeric material at the surface of the catheter tube is at least 5 dynes/cm greater than that of the lubricant.

10. The intermittent catheter of claim 1, wherein a surface energy of the polymeric material at the surface of the catheter tube is at least 10 dynes/cm greater than that of the lubricant.

11. An intermittent catheter, comprising:
a catheter tube of a polymeric material;
a hydrophilic coating disposed over a surface of at least a distal portion of the catheter tube;
a lubricant disposed over the hydrophilic coating, the lubricant configured to transition from a solid state into a liquid state upon insertion into a urethra of a patient or user; and
a funnel coupled to a proximal portion of the catheter tube, the funnel including a funnel opening in a proximal end of the funnel for voiding urine.

12. The intermittent catheter of claim 11, wherein the lubricant is disposed over the hydrophilic coating in a particulate form of the lubricant.

13. The intermittent catheter of claim 11, wherein the lubricant is substantially free of water.

14. The intermittent catheter of claim 11, wherein the lubricant has a melting point between 70° F. and 95° F.

15. The intermittent catheter of claim 11, wherein the lubricant is a natural fat, a synthetic fat, or a combination thereof.

16. The intermittent catheter of claim 11, wherein the lubricant is coconut oil, cocoa butter, palm oil, shea butter, or a mixture thereof.

17. The intermittent catheter of claim 11, wherein a surface energy of a surface of the hydrophilic coating is at least 5 dynes/cm greater than that of the lubricant.

18. The intermittent catheter of claim 11, wherein a surface energy of a surface of the hydrophilic coating is at least 10 dynes/cm greater than that of the lubricant.

19-40. (canceled)
